(19) United States
(12) Patent Application Publication
(10) Pub. No.: US 2009/0054906 A1
(43) Pub. Date: Feb. 26, 2009

(54) MEDICAL DEVICE AND METHOD FOR DELIVERING AN IMPLANT TO AN ANATOMICAL SITE

(75) Inventors: Ben Walthall, Austin, TX (US); Rodney E. Bristol, Cedar Park, TX (US); AnneMarie Gonin, Austin, TX (US)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(73) Assignee: ZIMMER ORTHOBIOLOGICS, INC., Austin, TX (US)

(21) Appl. No.: 12/196,831

(22) Filed: Aug. 22, 2008

Related U.S. Application Data
(60) Provisional application No. 60/957,827, filed on Aug. 24, 2007.

(51) Int. Cl. A61F 11/00; A61B 17/58; A61B 17/00

(52) U.S. Cl. 606/108; 606/99; 606/79

(57) ABSTRACT

A medical device and method for providing an implant to an anatomical site are disclosed. The medical device generally comprises a sleeve having first and second ends, a cutting frame coupled to the second end, and a rod received in an inner bore of the sleeve. The cutting frame defines a guide configured to direct a cutting tool for reciprocal motion along a cutting plane. When the implant is positioned within the inner bore, the rod and sleeve are used to gauge a depth of the anatomical site and to advance a portion of the implant beyond the cutting plane. The implant may then be cut to an appropriate size to fit the anatomical site.

### CROSS-REFERENCE

This application claims priority to pending U.S. Provisional Patent Application No. 60/957,827, filed Aug. 24, 2007 (pending), which is incorporated by reference herein in its entirety.

### TECHNICAL FIELD

Medical devices and methods, more particularly, medical devices and methods for gauging an appropriate size for an implant and delivering the appropriately sized implant to an anatomical site.

### BACKGROUND

Medical implants are often delivered to one or more anatomical sites within a patient's body to remedy anatomical defects. For example, a damaged bone within a patient's body may include a cavity or void formed by trauma, disease, surgery, etc. The cavity or void may render the bone prone to further injury or damage. To remedy such an anatomical defect, an implant is inserted into the cavity or void using a medical device. The implant may promote tissue formation, stabilize the defect, etc. Additionally, the implant may comprise healthy bone and/or cartilage harvested from a donor, or may comprise synthetic material(s) such as porous biocompatible foams and other soft polymers.

The extent to which the anatomical defect extends into the surrounding tissue can vary considerably. Typically, it is desirable that the length of the implant be about equal to the depth of the anatomical defect being treated. Such an arrangement helps ensure that the defect is effectively treated or occupied by the implant without portions of the implant significantly protruding beyond the surface of the tissue surrounding the defect. However, measuring the exact depth and shape of the anatomical defect to estimate the appropriate length of the implant can be challenging.

As a result, various devices have been developed to facilitate sizing an implant. For example, Leatherbury et al. U.S. Patent Application Publication No. 2004/0193154 discloses an implant delivery tool including an inner shaft disposed within a hollow outer shaft. The inner shaft has the same length as the outer shaft such that when an implant is inserted into a distal end of the outer shaft, the inner shaft is displaced beyond the proximal end of the outer shaft by a distance equal to the length of the implant. The protruding portion of the inner shaft is then inserted into the defect to be treated and the proximal end of the outer shaft is slid back over the inner shaft until the proximal end contacts the tissue surrounding the defect. Sliding the outer shaft in such a manner exposes a portion of the implant beyond the distal end of the outer shaft. Thus, the portion of the implant remaining within the outer shaft corresponds to the defect depth measured by the portion of the inner shaft protruding from the proximal end of the outer shaft.

Although Leatherbury teaches cutting off the protruding portion of the implant so that the remaining, appropriately sized portion can be delivered to the defect, the cutting techniques disclosed are not suited for implants that are relatively hard (by virtue of their material and/or density). For example, relatively hard implants require a significant amount of cutting force, and one technique simply uses a knife to cut the implant. Once the implant is sized, a surgeon removes the delivery tool from the defect and places it on a table with a sterile surface. The surgeon then steadies the outer shaft with one hand while cutting the protruding portion of the implant with a knife held in the other hand. Such a technique may suffice for implants that can be easily cut, such as synthetic implants constructed from biocompatible foams or polymers, but not for other types of implants, such as hard implants constructed from natural materials.

The Leatherbury device (OsteoBiologics, Inc. San Antonio Tex.) is marketed as part of a kit for TruFit™ implants. TruFit™ implants are bone graft substitute plugs constructed from porous, resorbable scaffolds of polymer materials. As a result, these implants can be easily cut with minimal force. Attempting to use the cutting technique discussed above for dense implants, such as allograft or xenograft implants constructed from bone and/or cartilage, can be difficult and have undesirable effects. Because a significant amount of cutting force is required to cut such implants, it is more difficult to both steady the outer shaft and guide the knife along a desired cutting plane during the cutting operation. This may result in the remaining portion of the implant having an uneven or angled surface that negatively affects how it fits within the measured defect. Moreover, although the distal end of the outer shaft may be used as a guide during the cutting operation, any contact between the knife and the outer shaft may generate debris that is ultimately delivered to the defect with the implant. Such debris has the potential to cause at least a localized inflammatory reaction.

Another cutting technique disclosed in Leatherbury uses a rectangular cutting base having a vertical cylindrical hole extending from the top to the bottom of the cutting base and a cutting slot horizontally extending from one side of the cutting base to intersect the vertical cylindrical hole. The cutting slot includes guides on opposite sides for guiding a cutting blade. Once the implant is sized, the distal end of the outer shaft of the delivery tool is inserted into the vertical cylindrical hole until the distal end contacts an internal shoulder above the horizontally extending slot. The cutting blade is then advanced linearly along the guides of the horizontally extending slot to cut the protruding portion of the implant.

Such a cutting technique may suffice for relatively soft, easily cut implants, but not for implants requiring significant cutting forces. Relatively hard implants must typically be cut by moving a cutting blade in a reciprocal manner numerous times, instead of simply pushing a blade in a single direction. Further, the rectangular cutting base disclosed in Leatherbury is bulky and not secured to the delivery tool in any manner. Maintaining the cutting base readily available but separate from the delivery tool while the implant is being sized may be an inconvenience. Additionally, the delivery tool must also be removed from the cutting base after the cutting operation so that the remaining portion of the implant may be pushed through the distal end and accurately delivered into the defect.

Thus, an improved medical device and method for sizing and cutting both soft and hard implants is desirable.

### SUMMARY

Medical devices and methods for delivering an implant to anatomical site. The described devices and methods are designed for sizing, cutting, and delivering an osteochondral implant or similar implant into a cavity in a bone, although the devices and methods may be used in connection with any other tissue and/or anatomical defects in a patient's body.

In one embodiment, the medical device generally comprises a sleeve having first and second ends, a cutting frame coupled to the second end, and a rod received in an inner bore defined by the sleeve. The cutting frame defines a guide configured to direct a cutting tool for reciprocal motion along a cutting plane. When the implant is positioned within the inner bore, an end portion of the rod extends beyond the first end of the sleeve. The rod is configured to advance a least a portion of the implant beyond the cutting plane when the sleeve is pushed at least partially over of the end portion of the rod.

In another embodiment, the medical device further comprises a delivery tube having an end removably coupled to at least one of the cutting frame or the second end of the sleeve. The delivery tube may be coupled to the cutting frame or sleeve after the implant has been cut to a desired size. The rod may then be used to advance the implant out of the sleeve and into the delivery tube, which may be subsequently removed from the cutting frame or sleeve. After positioning the delivery tube against the patient's body at the anatomical site, a delivery plunger may be received in the delivery tube to advance the implant through the delivery tube to the anatomical site.

In another embodiment, the medical device is provided as part of a kit that includes the implant. The kit may further include the cutting tool used to cut the implant to the desired size.

One method of using the medical device to provide an implant to an anatomical site generally comprises positioning at least a portion of the implant within the inner bore of the sleeve and inserting the rod into the inner bore. The rod is positioned so that the end portion extends beyond the first end of the sleeve. The sleeve is then pushed at least partially over the end portion of the rod to advance the implant past the cutting plane defined by the guide in the cutting frame. After directing a cutting tool in a reciprocal manner through the guide to cut the implant, the cut implant is pushed out of the sleeve and to the anatomical site.

### BRIEF DESCRIPTION OF THE DRAWINGS

The accompanying drawings, which are incorporated in and constitute a part of this specification, illustrate exemplary embodiments of the invention and, together with the summary given above, and the detailed description given below, serve to explain the invention.

FIG. 1 is an exploded perspective view showing an implant and a medical device according to one embodiment for delivering the implant to an anatomical site.

FIG. 2 is a perspective view showing the medical device of FIG. 1 being used to measure the depth of an anatomical defect in a bone.

FIGS. 3 and 4 are cross-sectional views showing a portion of the medical device of FIG. 1 being inserted into the anatomical defect of FIG. 2 to gauge the desired size of the implant.

FIG. 5 is a perspective view showing the implant being cut with a cutting tool.

FIG. 6 is a cross-sectional view showing the implant being cut in further detail.

FIG. 7 is a cross-sectional view showing the medical device of FIG. 1 being used to deliver the remaining portion of the implant to the anatomical defect after the implant is cut to the desired size and after a cutting frame is removed from the medical device.

FIG. 8 is an exploded perspective view showing an implant and a medical device according to another embodiment for delivering the implant to an anatomical site.

FIG. 9 is a perspective view showing the medical device of FIG. 8 with all components assembled.

FIG. 10 is a cross-sectional view showing the medical device of FIG. 8 with all components assembled after the implant has been cut to a desired size.

FIG. 11 is a cross-sectional view showing the medical device of FIG. 8 being used to transfer the implant from a sleeve to a delivery tube.

FIG. 12 is a cross-sectional view showing a delivery plunger being used to advance the implant through the delivery tube.

FIG. 13 is a cross-sectional view showing the delivery plunger being used to deliver the implant to an anatomical defect in a bone.

### DETAILED DESCRIPTION

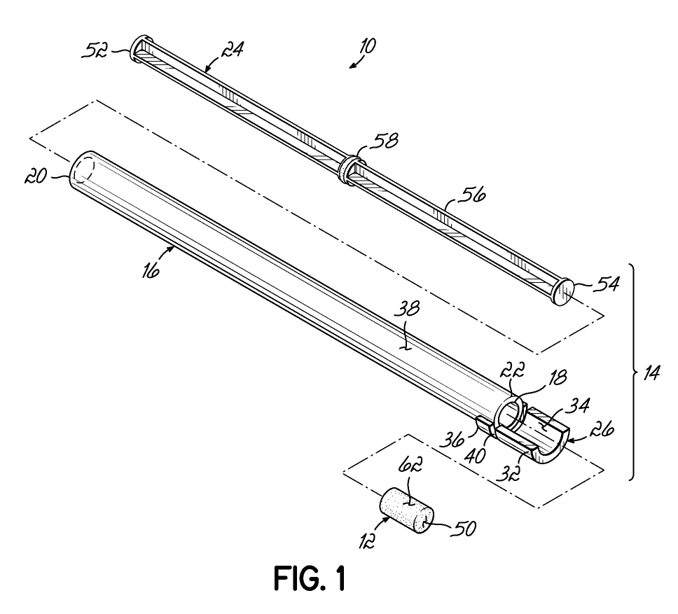

FIG. 1 shows one embodiment of a kit 10 including an implant 12 for a patient and a medical device 14 for providing the implant 12 to an anatomical site within the patient's body. The implant 12 may be any type structure for remedying a defect or to achieve some other desired function at the anatomical site. For example, the medical implant 12 may be an osteochondral allograft, autograft, and/or xenograft. It may form a plug configured to be inserted into an anatomical defect. In such situations, the medical device 14 may gauge and cut the implant 12 to a desired size, as will be described in greater detail below.

The medical device 14 includes a sleeve 16 defining an inner bore 18 extending from a first end 20 to a second end 22, a rod 24 received in the inner bore 18, and a cutting frame 26 coupled to the sleeve 16 at the second end 22. A variety of coupling arrangements may be used for the cutting frame 26 and sleeve 16. For example, the cutting frame 26 may be integrally formed with the sleeve 16 as part of a unitary structure, or may be a separate component secured to the sleeve 16 by adhesive, fasteners, or the like. Alternatively, the cutting frame 26 may be a separate component removably coupled to the sleeve 16 without the use of any fasteners.

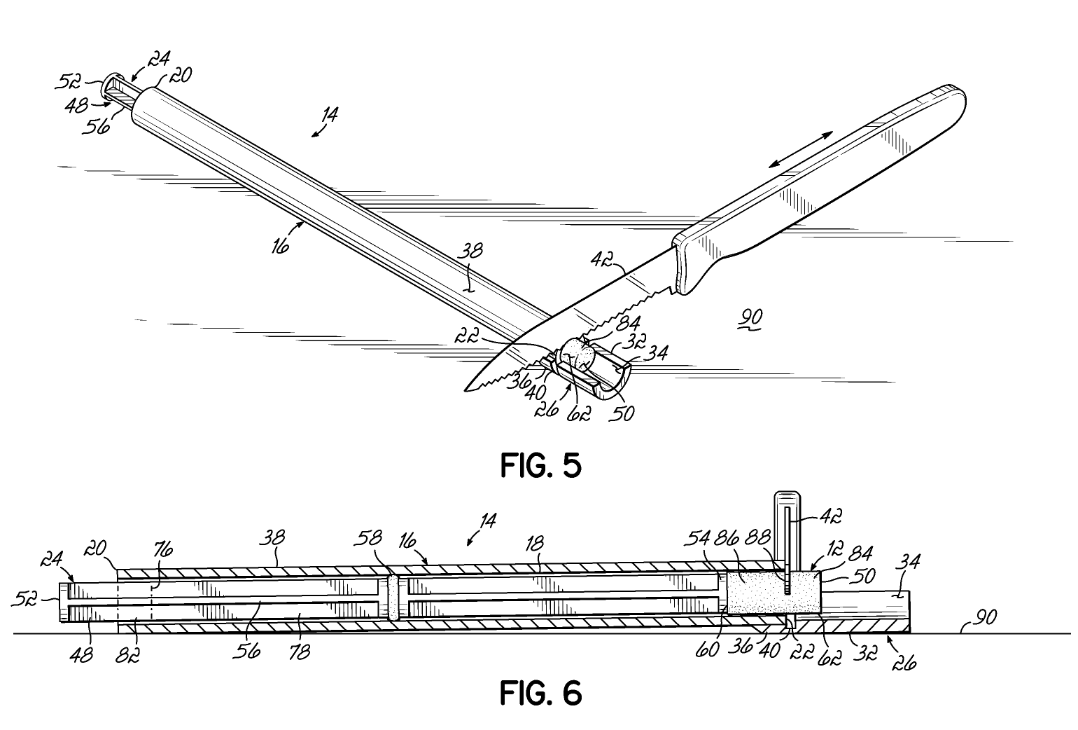

In the embodiment shown, the cutting frame 26 defines a trough having a distal portion 32 extending from the second end 22 of the sleeve 16 and a concave surface 34 substantially aligned with at least a portion of the inner bore 18. The cutting frame 26 may have a semi-circular profile and be configured to snap-fit onto the second end 22 of the sleeve 16. For example, a proximal portion 36 of the cutting frame 26 may have a profile substantially corresponding to an outer surface 38 of the sleeve 16, but sized so that it flexes and creates a frictional gripping force when pressed against the outer surface 38 at the second end 22. Alternatively, the proximal portion 36 may be a circular ring (not shown) designed to slide over the second end 22. Frictional forces between the proximal portion 36 and the second end 22 may be sufficient to secure the cutting frame 26 and prevent it from sliding along the sleeve 16 toward the first end 20. However, if desired, the sleeve 16 in either of these embodiments may further include a change in profile or protrusion (not shown) on the outer surface 38 to reduce or prevent such sliding movement. A slot or guide 40 provided in the cutting frame 26 between the proximal portion 36 and the distal portion 32 is configured to direct a cutting tool 42 (FIG. 5) for reciprocal motion along a cutting plane, as will be described in greater detail below.

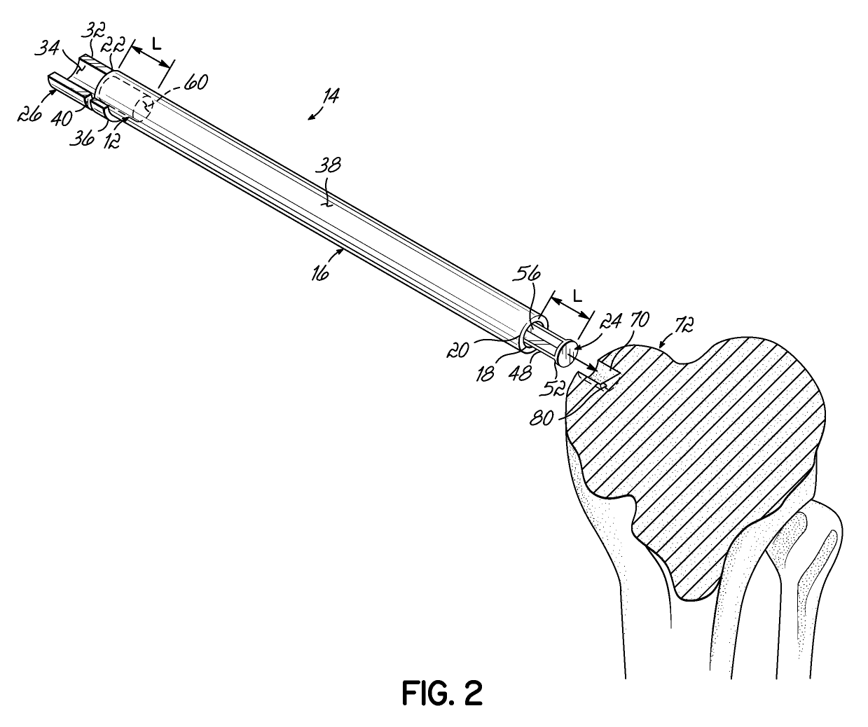

The inner bore 18 of the sleeve 16 is configured to receive both the rod 24 and the implant 12, as shown in FIG. 2. When at least a portion of the implant 12 is positioned within the inner bore 18, an end portion 48 of the rod 24 extends beyond the first end 20 of the sleeve 16. For example, in one embodiment, the rod 24 and the sleeve 16 are substantially the same length. Thus, when the implant 12 is positioned within the sleeve 16 so that a first end surface 50 of the implant 12 is substantially flush with the second end 22, the end portion 48 of the rod 24 extending beyond the first end 20 is about the same length as the implant 12. FIG. 2 illustrates the implant 12 and end portion 48 each having a length L.

The rod 24 and implant 12 may also have or be designed with the same diameter so that the end portion 48 further corresponds to the size of the implant 12. In one embodiment, the rod 24 includes a first end 52 and a second end 54 in the shape of a disc and an elongate element 56 extending between the first end 52 and second end 54. The elongate element 56 provides the rod 24 with a cross-shaped cross-sectional profile between the first end 52 and second end 54, although other cross-sectional configurations are possible. The rod 24 may further include at least one friction element 58 between the first end 52 and second end 54. The friction element 58 contacts an inner surface of the inner bore 18 to help stabilize the rod 24 within the sleeve 16, but still allows the rod 24 to slide relative to the sleeve 16. The rod 24 may also include a guide wire lumen (not shown) extending through its center for accommodating a guide wire used to direct the medical device 14 to an anatomical site.

One embodiment is a method of using the medical device 14 to gauge a desired size of the implant 12 and to provide the implant 12 to an anatomical site. The anatomical site described below is an anatomical defect, such as a void or cavity 70 (FIG. 2) within a bone 72. However, those skilled in the art will appreciate that the method may be used to provide one or more implants 12 to an anatomical site, or to provide one or more implants 12 to non-bone sites elsewhere in a patient's body.

As shown in FIG. 2, the implant 12 and rod 24 are first positioned within the sleeve 16. The implant 12 may have a diameter equal to or slightly larger than the diameter of the inner bore 18 so that a relatively small frictional force is created between an outer surface 62 of the implant 12 and the inner bore 18. This frictional force helps initially to retain the implant 12 within the sleeve 16 while the medical device 14 is manipulated to load the rod 24, or while the medical device 14 is moved from one area to another within an operating room. The rod 24 may be loaded into the sleeve 16 before or after the implant 12. If loaded before the implant 12, positioning the implant 12 within the sleeve 16 will cause the end portion 48 of the rod 24 to emerge from the sleeve 16 so as to extend from the first end 20. If loaded after the implant 12, the rod 24 is simply inserted into the inner bore 18 from the first end 20 until the rod 24 contacts a second end surface 60 of the implant 12. The end portion 48 of the rod 24 remains positioned outside of the inner bore 18 by a distance about equal to the length of the implant 12.

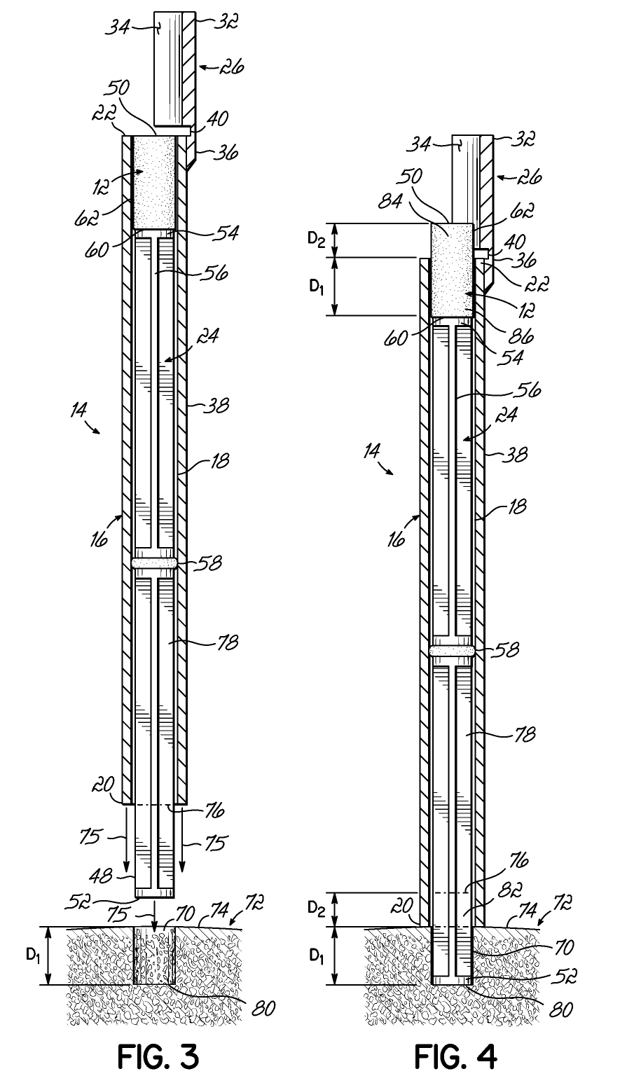

Once the implant 12 and rod 24 are positioned within the sleeve 16, the medical device 14 is moved toward the bone 72 so that the end portion 48 of the rod 24 may be inserted into the void or cavity 70. FIGS. 3 and 4 illustrate this aspect in further detail. The first end 20 of the sleeve 16 is first positioned proximate the bone 72 and the end portion 48 of the rod 24 is aligned with the void or cavity 70, which has a depth D1. The sleeve 16 and rod 24 are then moved together in the direction of arrows 75 so that the end portion 48 is received in the void or cavity 70. The dashed line 76 represents the boundary between the end portion 48 and a remainder 78 of the rod 24. When the first end 52 of the rod 24 contacts a bottom surface 80 of the void or cavity 70 or cannot otherwise be advanced further within the void or cavity 70, a top part 82 of the end portion 48 may remain outside the void or cavity 70. The surgeon may then continue to move the sleeve 16 toward the bone 72 by sliding or pushing the sleeve 16 along the rod 24 until the second end 22 contacts tissue 74 surrounding the void or cavity 70.

By the time the first end 20 contacts the tissue 74, the sleeve 16 has been advanced over the end portion 48 of the rod 24 by a distance D2. The presence of the rod 24 within the inner bore 18 prevents the implant 12 sliding along with the sleeve 16 during this relative movement between the sleeve 16 and rod 24. As a result, when the sleeve 16 is advanced over the rod 24 by the distance D2, the sleeve 16 is also advanced over the implant 12 by the distance D2 so that an excess portion 84 of the implant 12 extends beyond the first end 20. A remaining portion 86 of the implant 12 within the inner bore 18 then has a length that is about equal to the depth D1. To this end, the remaining portion 86 represents an appropriate size of the implant 12 for effectively being positioned within the void or cavity 70.

Once the appropriate size of the implant 12 has been gauged, the excess portion 84 may be cut off from the remaining portion 86. In one embodiment, the medical device 14 is moved away from the bone 72 and placed on a surface such as a nearby table (not shown) or other sterile surface 90 (FIG. 5) without sliding the outer sleeve 16 relative to the rod 24. The surgeon may then cut the excess portion 84 along the cutting plane defined by the guide 40 with a cutting tool 42, as shown in FIGS. 5 and 6, while gripping the sleeve 16 with one hand to stabilize the medical device 14.

The guide 40 on the cutting frame 26 helps maintain the cutting tool 42 in the cutting plane when the cutting tool 42 is moved in a reciprocal manner, as is typically required for relatively hard implants. For example, if the implant 12 is constructed from natural bone or cartilage tissue, an effective way to cut the implant 12 may be to move the cutting tool 42 repeatedly back and forth over and in contact with the implant 12 in a direction generally transverse to the implant 12. Although significant cutting forces may be required that make it difficult to control the orientation and/or position of the cutting tool 42, the guide 40 prevents the cutting tool 42 from moving out of the cutting plane. Instead, the cutting tool 42 moves back and forth through the guide 40 to form a substantially flat end surface 88 on the remaining portion 86.

The cutting tool 42 in FIGS. 5 and 6 is a knife moved manually by the surgeon. However, those skilled in the art will appreciate that a wide variety of other cutting tools or arrangements may be used to cut the implant 12. Examples include but are not limited to: saws, chisels, electrocautery devices, and machines incorporating a blade. For example, because the cutting frame 26 allows for reciprocal motion, the cutting tool 42 may alternatively be a blade (not shown) driven in a reciprocal manner by a machine or power tool. The medical device 14 may still be stabilized by hand in such embodiments or may be secured to by a clamp (not shown) or the like to the machine or some other stable structure.

After cutting the implant 12 to the appropriate size for the void or cavity 70, the cutting frame 26 may be removed from the second end 22 of the sleeve 16. In embodiments where the cutting frame 26 is coupled to the sleeve 16 by fasteners (not shown), the cutting frame 26 may be released from the sleeve 16 by removing the fasteners. In embodiments where the cutting frame 26 is snap-fit or otherwise frictionally coupled to the sleeve 16, the cutting frame 26 may be pulled away from the sleeve 16. Thus, the snap-fit or frictional connection is sufficiently secure to keep the cutting frame 26 coupled to the sleeve 16 while the cutting tool 42 is directed through the guide 40, but may still allow for easy removal of the cutting frame 26 when the cutting frame 26 is pulled in a particular manner.

In other embodiments, at least a portion of the cutting frame 26 may be broken off to effectively remove the cutting frame 26 from the sleeve 16. For example, once the cutting operation is complete, the distal portion 32 of the cutting frame 26 may be broken off from the proximal portion 36. The guide 40 between the proximal portion 36 and distal portion 32 may create a weak area in the cutting frame 26 that allows it to be broken in such a manner. Thus, the guide 40 may serve as a frangible connection between the proximal portion 36 and the distal portion 32. With only the proximal portion 36 of the cutting frame 26 remaining coupled to the sleeve 16, no portion of the cutting frame 26 extends beyond the second end 22. It will be appreciated that the cutting frame 26, and particularly the proximal portion 36, may incorporate additional or alternative frangible connections to achieve this same arrangement (after the cutting frame 26 is removed). Additionally, the excess portion 84 of the implant 12 may be removed along with the cutting frame 26 or prior to the removal of the cutting frame 26.

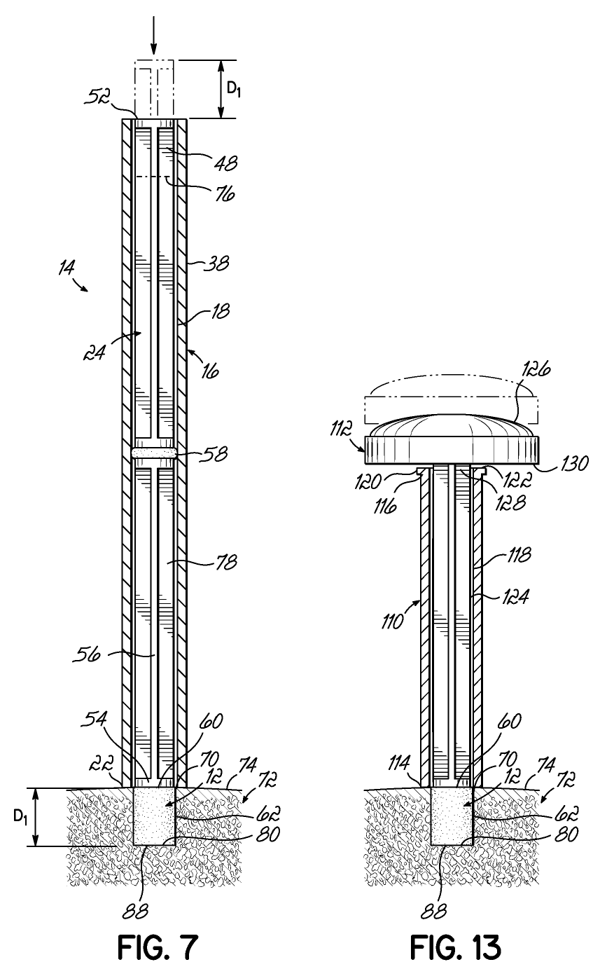

As shown in FIG. 7, the second end 22 of the sleeve 16 (with the cutting frame 26 removed) may next be positioned against the tissue 74 with the inner bore 18 aligned with the void or cavity 70. To provide the implant 12 to the void or cavity 70, the surgeon pushes the end portion 48 of the rod 24 that extends beyond the first end 20 into the inner bore 18. The rod 24, in turn, slides through the sleeve 16 to advance the implant 12 beyond the second end 22 and into the void or cavity 70. By the time the first end 52 of the rod 24 is substantially flush with the first end 20 of the sleeve 16, the implant 12 has been completely advanced out of the inner bore 18 and into the void or cavity 70. The second end surface 60 of the implant 12 is substantially flush with the tissue 74 because the implant 12 has been appropriately sized to effectively fill the void or cavity 70.

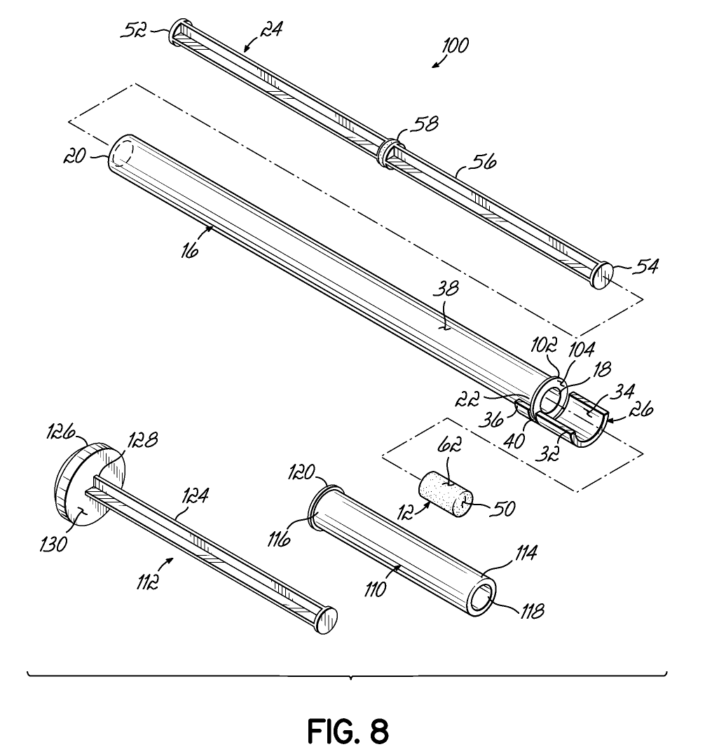

FIG. 8 illustrates a kit 100 according to an alternative embodiment. Because the kit 100 includes components similar to those in the kit 10 discussed above, like reference numbers are used to refer to like structure from the kit 10. Specifically, the kit 100 includes the implant 12, the sleeve 16, the cutting frame 26, and the rod 24. The sleeve 16 may further include a flange 102 radially extending from the outer surface 38 at the second end 22 to define an end surface 104. Although the flange 102 may be received in the guide 40 of the cutting frame 26, its presence does not affect the sizing and cutting of the implant 12. In particular, the sleeve 16, rod 24, and cutting frame 26 may be used to gauge the depth of an anatomical defect in the same manner discussed above with reference to FIGS. 2-6 so that the implant 12 may be cut with the cutting tool 42 (FIG. 5) to an appropriate or desired size. The anatomical defect may be, for example, the void or cavity 70 (FIG. 2) in bone 72.

In the kit 100, the cutting frame 26 does not need to be removed from the sleeve 16 to deliver the implant 12 because the kit 100 further includes a delivery tube 110 and delivery plunger 112. The delivery tube 110 includes a first end 114, a second end 116, and an inner bore 118 extending between the first end 114 and second end 116. A flange 120 provided on the delivery tube 110 at the second end 116 defines an end surface 122 (FIG. 10), similar to the flange 102 on the sleeve 16. The delivery plunger 112 includes a shaft 124 that may be constructed in a manner similar to the rod 24 and a handle 126 coupled to an end 128 of the shaft 124. Although the handle 126 is shown as a dome-shaped structure having a bottom surface 130, those skilled in the art will appreciate that a wide variety of other shapes and configurations are possible.

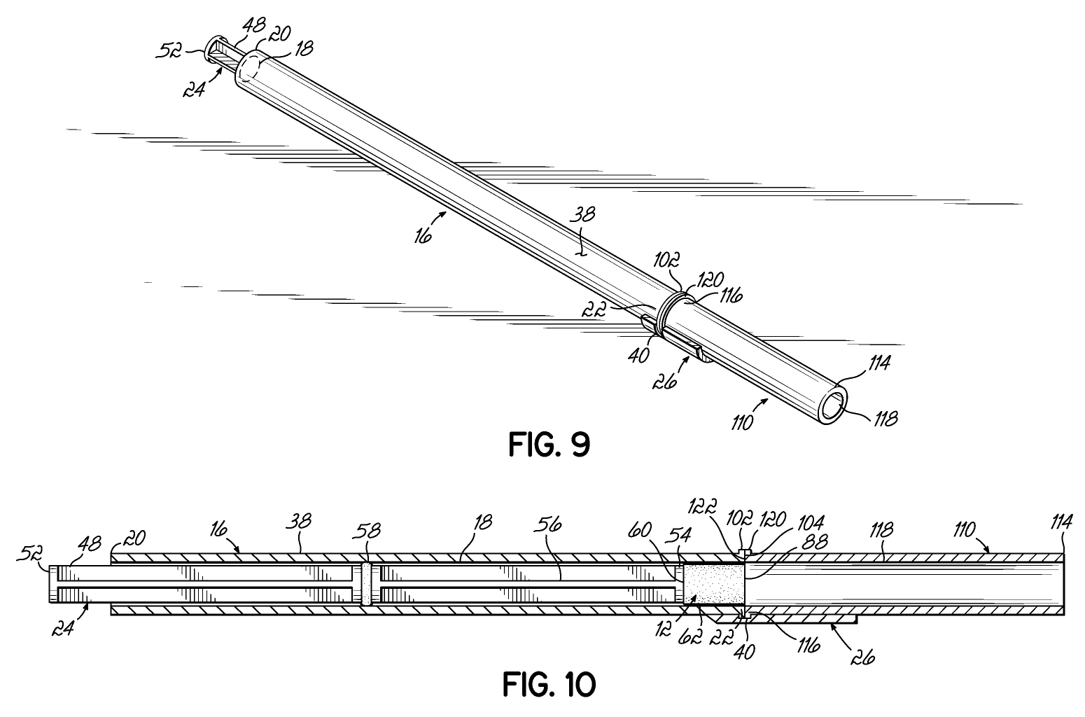

As shown in FIGS. 9 and 10, the delivery tube 110 may be coupled to the cutting frame 26 and aligned with the sleeve 16 after the implant 12 has been cut using the cutting tool 42 (FIG. 5). For example, the delivery tube 110 may be positioned on the cutting frame 26 with the flange 120 received in the guide 40. The flange 120 mates with the flange 102 so that the end surface 122 and end surface 104 confront each other. Such an arrangement aligns the inner bore 18 of the sleeve 16 with the inner bore 118, which may have a diameter approximately equal to that of the inner bore 18 and/or implant 12. If desired, the delivery tube 110 may be coupled to the second end 22 of the sleeve 16 instead of, or in addition to, being coupled to the cutting frame 26 using any suitable coupling arrangement.

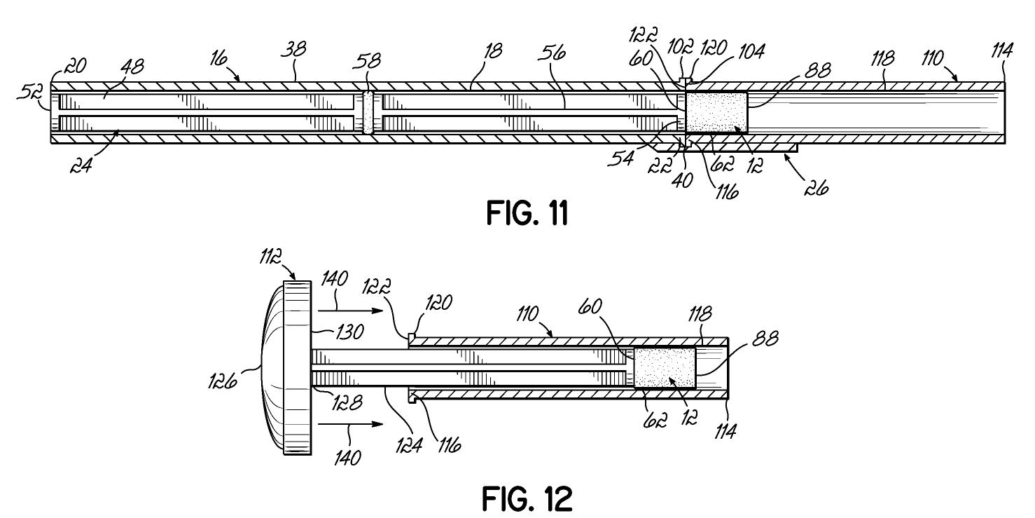

Once the delivery tube 110 is aligned with the sleeve 16, the surgeon pushes the end portion 48 of the rod 24 extending from the first end 20 of the sleeve 16 into the inner bore 18 to force the implant 12 out of the second end 22 and into the delivery tube 110. FIG. 11 illustrates the components of the kit 100 after the implant 12 has been transferred to the delivery tube 110. With the implant 12 now positioned in the inner bore 118, the surgeon may then remove the delivery tube 110 from the cutting frame 26. The delivery tube 110 and delivery plunger 112, rather than the sleeve 16 and rod 24, may then be used to deliver the implant 12 to the desired anatomical site.

For example, as shown in FIG. 12, the shaft 124 of the delivery plunger 112 may be inserted into the inner bore 118 from the second end 116 of the delivery tube 110. Pushing the handle 126 in the direction of arrows 140 causes the shaft 124 to advance the implant 124 toward the first end 114. The shaft 124 may be slidably received in the delivery tube 110 to provide stability and to facilitate advancing the implant 12. Once the implant 12 is advanced an initial distance, the first end 114 of the delivery tube 110 may be positioned against the tissue 74 surrounding the void or cavity 70 in the bone 72, as shown in FIG. 13. The handle 126 of the delivery plunger 112 is then pushed toward the delivery tube 110 to further advance the shaft 124 and to deliver the implant 12 into the void or cavity 70. Alternatively, the first end 114 may be positioned against the tissue 74 before inserting the shaft 124 of the delivery plunger 112 into the inner bore 118.

In one embodiment, the shaft 124 and the delivery tube 110 are substantially the same length. Thus, the bottom surface 130 of the handle 126 will contact the end surface 122 of the delivery tube 110 once the shaft 124 reaches the first end 114 of the delivery tube 110. This contact provides the surgeon with a tactile indication that the implant 12 has been delivered out of the delivery tube 110 and into the void or cavity 70.

While the invention has been illustrated by the description of various embodiments, and while the various embodiments have been described in considerable detail, the inventors do not intend to restrict or in any way limit the scope of the appended claims to such detail. Additional advantages and modifications will readily appear to those skilled in the art. For example, instead of being positioned immediately adjacent the second end 22 of the sleeve 16, the guide 40 may alternatively be spaced from the second end 22. In such an embodiment, the rod 24 may have a length equal to the length of the sleeve 16 plus the distance between the guide 40 and the second end 22. Moreover, the various features of the invention may be used alone or in numerous combinations depending on the needs and preferences of the user. For example, the sleeve 16 in the kit 10 may also include the flange 102 at the second end 22 even though the delivery tube 110 may not be used in the kit 10. Therefore, the invention in its broader aspects is not limited to the specific details, representative apparatus and methods, and illustrative examples shown and described. Accordingly, departures may be made from such details without departing from the scope or spirit of the general inventive concept.

What is claimed is:

1. A medical device for delivering an implant to an anatomical site, comprising:
a sleeve having a first end and a second end, the sleeve defining an inner bore configured to receive the implant;
a rod received in the inner bore, the rod including an end portion configured to extend beyond the first end of the sleeve when at least a portion the implant is positioned within the inner bore; and
a cutting frame coupled to the second end of the sleeve, the cutting frame defining a guide configured to direct a cutting tool for reciprocal motion along a cutting plane;
wherein the rod is configured to advance a least a portion of the implant beyond the cutting plane when the sleeve is pushed at least partially over of the end portion of the rod.

2. The device of claim 1, wherein the rod and the sleeve are substantially the same length.

3. The device of claim 2, wherein the end portion of the rod and the implant are substantially the same length.

4. The device of claim 1, wherein the end portion of the rod and the implant have substantially the same diameter.

5. The device of claim 1, wherein the cutting frame includes a concave surface substantially aligned with at least a portion of the inner bore of the sleeve.

6. The device of claim 5, wherein the concave surface has a substantially semi-circular profile.

7. The device of claim 5, wherein the cutting frame defines a trough extending from the second end of the sleeve and including the concave surface, the guide being defined by a slot provided in the trough.

8. The device of claim 7, wherein the sleeve is aligned along a longitudinal axis, the slot being aligned in a plane substantially perpendicular to the longitudinal axis.

9. The device of claim 1, wherein at least a portion of the cutting frame is removably coupled to the second end of the sleeve.

10. The device of claim 1, wherein the rod is slidably received in the inner bore.

11. The device of claim 1, further comprising:
a delivery tube having an end removably coupled to at least one of the cutting frame or the second end of the sleeve, wherein the rod is configured to advance the implant out of the sleeve and into the delivery tube.

12. The device of claim 11, further comprising:
a delivery plunger configured to be received in the delivery tube to advance the implant through the delivery tube.

13. The device of claim 12, where in the delivery plunger is configured to slide relative to the delivery tube.

14. A kit for repairing an anatomical site, the kit comprising:
an implant; and
a delivery device including:
a sleeve having a first end and a second end, the sleeve defining an inner bore in which at least a portion of the implant is received;
a rod received in the inner bore of the sleeve, the rod including an end portion extending beyond the first end of the sleeve; and
a cutting frame coupled to the second end of the sleeve, the cutting frame defining a guide configured to direct a cutting tool for reciprocal motion along a cutting plane;
wherein the rod advances at least a portion of the implant beyond the cutting plane when the sleeve is pushed over at least a portion of the end portion of the rod.

15. The kit of claim 14, further comprising:
a cutting tool for cutting the implant to a desired size.

16. The kit of claim 14, wherein the anatomical site is at a cavity within a bone.

17. The kit of claim 14, wherein the implant is substantially cylindrical.

18. The kit of claim 14, wherein the implant comprises a natural material.

19. The kit of claim 18, wherein the implant comprises at least one of an osteochondral allograft, an osteochondral autograft, or an osteochondral xenograft.

20. The kit of claim 14, wherein the implant comprises a synthetic material.

21. The kit of claim 14, wherein the rod and the sleeve are substantially the same length.

22. The kit of claim 21, wherein the end portion of the rod and the implant are substantially the same length.

23. The kit of claim 14, wherein the end portion of the rod and the implant have substantially the same diameter.

24. The kit of claim 14, wherein at least a portion of the cutting frame is removably coupled to the second end of the sleeve.

25. The kit of claim 14, wherein the rod is slidably received in the inner bore of the sleeve.

26. A method of delivering a medical implant to an anatomical site, the method comprising:
positioning at least a portion of the implant within an inner bore of a sleeve, the sleeve having a first end and a second end;
inserting a rod into the inner bore of the sleeve;
positioning the rod so that an end portion of the rod extends beyond the first end of the sleeve;
pushing the sleeve at least partially over the end portion of the rod to advance the implant along a cutting frame coupled to the second end of the sleeve, at least a portion of the implant being advanced past a cutting plane defined by a guide on the cutting frame;
directing a cutting tool in a reciprocal manner through the guide to cut the implant along the cutting plane; and
pushing the cut implant out of the sleeve and to the anatomical site.

27. The method of claim 26, wherein positioning the implant at least partially within the inner bore further comprises:
positioning the implant within the inner bore so that an end of the implant is substantially flush with the second end of the sleeve.

28. The method of claim 27, wherein positioning the implant within the inner bore results in the end portion of the rod extending beyond the first end of the sleeve by a distance approximately equal to the length of the implant.

29. The method of claim 26, wherein the anatomical site is at a cavity within a bone.

30. The method of claim 29, wherein pushing the sleeve at least partially over the end portion of the rod further comprises:
inserting the end portion of the rod into the cavity of the bone until the end portion contacts a bottom region of the cavity; and
advancing the sleeve over the end portion until the sleeve contacts a surface of the bone around the cavity.

31. The method of claim 30, further comprising:
removing at least a portion of the cutting frame from the second end of the sleeve after the implant is cut with the cutting tool, wherein at least a portion of the implant remains within the inner bore of the sleeve;
removing the end portion of the rod from the cavity and the first end of the sleeve from the surface of the bone;
positioning the second end of the sleeve against the surface of the bone around the cavity; and
pushing the end portion of the rod within the sleeve to force the implant out of the second end and into the cavity.

32. The method of claim 30, further comprising:
coupling a delivery tube to at least one of the cutting frame or the second end of the sleeve;
pushing the end portion of the rod within the sleeve to force the implant out of the second end into the delivery tube;
positioning an end of the delivery tube against the surface of the bone around the cavity;
inserting a delivery plunger into the delivery tube to advance the implant through the delivery tube into the cavity.
